(19) United States
(12) Patent Application Publication
(10) Pub. No.: US 2015/0204886 A1
(43) Pub. Date: Jul. 23, 2015

(54) TROPONIN I (TnI) AS A SUITABLE MARKER PROTEIN FOR THE DETERMINATION OF ANIMAL SPECIES ORIGIN OF ADIPOSE TISSUE

(71) Applicant: FLORIDA STATE UNIVERSITY RESEARCH FOUNDATION, TALLAHASSEE, FL (US)

(72) Inventor: Yun-Hwa Peggy Hsieh, Tallahassee, FL (US)

(21) Appl. No.: 14/421,289

(22) PCT Filed: Aug. 26, 2013
(86) PCT No.: PCT/IB2013/056896

§ 371 Date: Feb. 12, 2015

Related U.S. Application Data
(60) Provisional application No. 61/693,811, filed on Aug. 28, 2012.

(51) Int. Cl. G01N 33/68

(52) U.S. Cl. CPC G01N 33/6887; G01N 2333/4703

(57) ABSTRACT

A method for determining the species of adipose tissue based on the species of troponin I (TnI) detected in the adipose tissue is described.

### CROSS-REFERENCE TO RELATED APPLICATIONS

This application claims benefit of priority to U.S. Provisional Patent Application No. 61/693,811 filed Aug. 28, 2012, entitled “TROPONIN I (TNI) AS A SUITABLE MARKER PROTEIN FOR THE DETERMINATION OF ANIMAL SPECIES ORIGIN OF ADIPOSE TISSUE”, which is incorporated herein by reference in its entirety.

### BACKGROUND

1. Field of the Invention

The present invention relates to a method for determining the animal species origin of adipose tissue.

2. Related Art

Species origin of animal fat and fat tissues can be identified by several fat-based and DNA-based methods including gas chromatography, FT-Raman spectroscopy, Fourier transform infrared (FTIR) spectroscopy and near infrared (NJR), and PCR techniques. All of these instrumental methods require the use of a pure lipid sample or involve laborious extraction of fatty acids or DNA from the sample product, use of sophisticated instruments, complicated data processing and interpretation. These methods are effective only if the analyzed fat sample is present in copious amounts and from a single species. However, it becomes very difficult to interpret data for the identification of species origin from a mixed sample which contains fats derived from two or more species, and from processed food.

### SUMMARY

According to a first broad aspect, the present invention provides a method comprising the following steps: (a) determining a species of a sample of adipose tissue based on a species of troponin I (TnI) detected in the sample of adipose tissue, and (b) displaying the species of the sample of adipose tissue determined in step (a) to a user on a visual display device and/or saving the species of the sample of adipose tissue determined in step (a) to a storage medium.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 is an SDS-PAGE protein profile of cooked and raw pork meat and fat extracts with and without heating treatment.

FIG. 2 is an SDS-PAGE protein profile of cooked and raw beef meat and fat extracts with and without heating treatment.

FIG. 3 is an SDS-PAGE protein profile of cooked and raw chicken meat and fat extracts with and without heating treatment.

FIG. 4 is a western blot showing the species-specificity of MAb 5H9 for meat and fat extracts from different species.

FIG. 5 is a western blot showing the species-specificity of 1B2 for meat and fat extracts from different species.

FIG. 6 is a western blot showing the species-specificity of 2G3 for meat and fat extracts from different species.

FIG. 7 shows a western blot result for the antigenic protein of MAb 8D3.

FIG. 8 shows a western blot result for the antigenic protein of MAb 6B 11.

FIG. 9 shows a western blot result for the antigenic protein of MAb 5A2.

FIG. 10 shows a western blot result for the antigenic protein of MAb 7G7.

FIG. 11 shows a western blot result for the antigenic protein of MAb 4D3.

FIG. 12 shows a western blot analysis of a fat extract based on MAb 5B5.

FIG. 13 shows a western blot analysis of a meat extract based on MAb 5B5.

### DETAILED DESCRIPTION OF THE PREFERRED EMBODIMENTS

### Definitions

Where the definition of a term departs from the commonly used meaning of the term, applicant intends to utilize the definitions provided below, unless specifically indicated.

For purposes of the present invention, it should be noted that the singular forms, “a,” “an” and “the” include reference to the plural unless the context as herein presented clearly indicates otherwise.

For purposes of the present invention, directional terms such as “top,” “bottom,” “upper,” “lower,” “above,” “below,” “left,” “right,” “horizontal,” “vertical,” “up,” “down,” etc., are merely used for convenience in describing the various embodiments of the present invention. The embodiments of the present invention may be oriented in various ways. For example, the diagrams, apparatuses, etc., shown in the drawing figures may be flipped over, rotated by 90° in any direction, reversed, etc.

For purposes of the present invention, a value or property is “based” on a particular value, property, the satisfaction of a condition, or other factor, if that value is derived by performing a mathematical calculation or logical decision using that value, property or other factor.

For purposes of the present invention, the term “computer” refers to any type of computer or other device that implements software including an individual computer such as a personal computer, laptop computer, tablet computer, mainframe computer, mini-computer, etc. The term “computer” also refers to electronic devices such as an immunoassay system, scanner, a sensor, smartphone, an eBook reader, a cell phone, a television, a handheld electronic game console, a videogame console, a compressed audio or video player such as an MP3 player, a Blu-ray player, a DVD player, a microwave oven, etc. In addition, the term “computer” refers to any type of network of computers, such as a network of computers in a business, a computer bank, the Cloud, the Internet, etc. In one embodiment of the present invention, a computer may be employed to control the performance of one or more steps of the method of the present invention and/or to conduct one or more steps of the present invention.

For purposes of the present invention, the term “sample of adipose tissue” refers to a sample comprising adipose tissue. Other types of tissue, contaminants and materials such as muscle, blood, etc. may be present in a sample of adipose tissue.

For purposes of the present invention, the term “species” refers to a species of an animal and to the name of the species. For example, in one embodiment of the present invention, the “species,” i.e., the name of the species, of a sample may be displayed to a user and/or saved to a storage medium.

For purposes of the present invention, the term “species of a sample of adipose tissue” refers to the species of the animal(s) that is (are) the source of the adipose tissue of the sample. In one embodiment, the present invention may be used to detect different animal species in a sample of adipose tissue when using species-specific TnI. For example, bovine-specific TnI antibody may be used to detect bovine fat, porcine-specific TnI antibody may be used to detect porcine fat, etc. Therefore, in some embodiments of the present invention, the “species of a sample of adipose tissue” may be two or more species.

For purposes of the present invention, the term “species of TnI” refers to the TnI for a specific species.

For purposes of the present invention, the term “storage medium” refers to any form of storage that may be used to store bits of information. Examples of storage include both volatile and non-volatile memories such as MRRAM, MRRAM, ERAM, flash memory, RFID tags, floppy disks, Zip™ disks, CD-ROM, CD-R, CD-RW, DVD, DVD-R, flash memory, hard disks, optical disks, etc.

For purposes of the present invention, the term “visual display device,” the term “visual display apparatus” and the term “visual display” refer to any type of visual display device or apparatus such as a an LCD screen, touchscreen, a CRT monitor, LEDs, a projected display, a printer for printing out an image such as a picture and/or text, etc. A visual display device may be a part of another device such as a spectrometer, a computer monitor, a television, a projector, a cell phone, a smartphone, a laptop computer, a tablet computer, a handheld music and/or video player, a personal data assistant (PDA), a handheld game player, a head mounted display, a heads-up display (HUD), a global positioning system (GPS) receiver, etc. In one embodiment of the present invention, a visual display device may be employed to display to a user the results of one or more steps of the method of the present invention and/or the progress of one or more steps of the method of present invention.

### Description

A recent study reported that only DNA and a protein-based immunoassay could determine the species (ruminant) content of fat in meat and bone meals. DNA-based methods usually are ineffective against samples that have undergone processes such as severe heat processing (e.g., canning) and hydrolysis, which damages DNA and hence reduces the yield and quality of the amount of DNA extracted from such processed foods samples. Besides, both fat-based and DNA-based methods have focused almost exclusively on the detection of animal fat in raw samples and hence cannot be guaranteed to be equally effective against heat-processed counterparts. Rapid and effective methods for the determination of fat species in a mixture have not been reported in the literature although such methods are urgently needed.

Immunoassays based on the specific antibody-antigen recognition have been widely accepted as a simple, rapid and specific analytical technique for agricultural and food analyses, either qualitatively or quantitatively. Usually the assay can be performed in a complicated sample mixture without laborious isolation or purification of the target analyte(s) from the sample. In order to develop an immunoassay for rapid species content determination, one critical element is the availability of species-specific antibodies as the probe to recognize the analyte (antigen). A species marker thus should firstly be identified in the adipose tissue which can be used as the target analyte for the antibody development. Most proteins are heat-labile and become insoluble after heating to certain degree. The conditions of an ideal species marker should be (1) that the antigen marker is present in the tissue in significant amount and is uniformly distributed throughout the tissue so that the detection result can be sensitive and representative, and (2) that the binding between the antibody and the antigen is stable after heat processing so that cooking would not affect the immunoreactivity for the detection.

In one embodiment, the present invention employs a universal and heat-stable muscle protein, troponin I (TnI), as a species marker protein in the adipose tissue. TnI is a ˜23 KDa subunit protein of the myofibril protein “troponin.” Although the presence of a number of proteins has been reported in animal fat tissue, the presence of TnI in the animal adipose tissue has now been discovered. Furthermore, TnI may be used for fat speciation. Because this protein has species-specific amino acid sequence regions, antibodies developed against this protein can be species specific if the binding site (epitope) is located at the species-specific region of the peptide. Such antibodies, including monoclonal or polyclonal antibodies, thus would be suitable to be used in an immunoassay to identify animal species not only in muscle but also in adipose tissues. While application of TnI as a species marker protein for the meat species identification has been reported in the literature, the use of TnI as a species marker for the species analysis of fat tissue has never been reported.

Immunoassays based on the detection and quantification of this marker protein are able to reliably, sensitively and rapidly detect animal species (pork, beef, poultry, etc.) in fat-in-fat or fat-in-meat mixtures at low levels (˜1% w/w). Also, simplified protein extraction methods from the adipose tissue have been developed. These simple methods only require aqueous extraction without homogenization of the sample admixture, although require a mild heat treatment. These methods will facilitate the analyses of variations of immunoassay in terms of time and costs. With the discovery of the fact that TnI can serve as a heat-stable species-marker in adipose tissue combined with the developed simple sample extraction methods, the application of TnI-based immunoassays for a rapid species identification and species content determination of animal fat in both raw and heat-processed samples can be accomplished. The success of this new application may be demonstrated by using several previously developed anti-TnI antibodies (porcine-specific, bovine-specific and all animal-specific) in several variations of immuuoassays (ELISA, western blot and lateral flow strip assay). There has never been any protein-based immunoassay reported in the literature for the rapid determination of species content of animal fat, especially it can be rapidly (minutes to few hours) done in either raw or cooked products with a low detection limit (approximately 1%).

### Applications

Effective rapid methods for the species determination of fat tissue in a sample admixture are lacking but they are urgently needed. For example, hidden or fraudulent use of pork fat in a variety of food products to improve the texture, flavor or boost the bulk of the final product is an affront not only to Jews and Muslims who by the dictates of their religion are forbidden to consume anything derived from pig, it also violates the domestic and international food labeling laws. On the other hand, ruminant (cattle, deer, sheep and goat) proteins are banned in ruminant animal feed worldwide for the prevention of fatal prion diseases (mad cow disease and human Creutzfeldt-Jakob disease). Contamination of any ruminant tissue including adipose tissue would impose risks of transmitting prions from infected animals. Furthermore, in recent times there is a preference to use vegetable oil in place of animal fat in food processing because of the unhealthy fatty acid profile of animal fat. Among animal fats, pork and beef fats are most commonly used. Accordingly, the use of pork or beef fat, which traditionally had been the choice of fat for deep frying because they are cheap and stable, is restricted to only foods where its unique flavor is desirable. Unfortunately, adulteration of vegetable oils with animal fat in the formulation of shortenings, margarines and other specialty food oils is a common practice. Therefore, rapid methods for the sensitive detection of target materials in raw, cooked or rendered products are desired for consumer protection. Currently, immunoassay kits for the species identification of muscle tissue are available commercially (ELISA Technologies Inc., Neogen Co.) However, these assays were not designed and cannot detect the presence of target fat tissue in the sample.

A series of thermal-stable, species-specific antibodies may be used for the detection of a number of animal proteins such as tropomyosin, troponin, myosin, sarcoplasmic proteins, blood cellular and serum proteins in raw and cooked products. It has now been found that substantial amounts of proteins can be extracted from muscle-free adipose tissue even after cooking. Adipose tissue typically contains about 2% proteins.

In animal fat products about 0.15% are insoluble impurities, about 85% of which is proteinaceous. Among these proteins, it is possible to identify the thermal-stable 23 KDa TnI to be the most suitable antigenic protein in adipose tissue for species-specific antibody development. Any immunoassays using species-specific anti-TnI antibodies with the thermal-stable epitopes can now be used not only for speciation in muscle samples but also in fat tissue and products, both raw and cooked. However, the new sample extraction methods should be employed to perform the appropriate immunoassay.

Fat Species Adulteration has been a Widespread Problem

Oils and fats have long played an important role as an essential nutrient in the human diet and are derived either from plant or animal sources. Adipose tissue of livestock animals is a major by-product obtained from meat processing and is often used as an ingredient in meat and food products (Aida et al. 2007 (Reference 5); Abbas et al. 2009 (Reference 1)). Among animal fats, pork and beef fats are most commonly used. Pork fat has been more widely used in meat and food industries to improve the texture, flavor and/or boost weight. However, food containing ingredients derived from a porcine source may cause serious concerns in the view of some religions, such as Islam and Judaism, and for vegetarians. Adulterating vegetable oils with tallow may present a health risk as the possibility of tallow carrying the infectious agent-prion that causes transmissible spongiform encephalopathy (TSE) has been reported (ECSSC 1999 (Reference 20). Adulteration with less valuable or undeclared meat or fat species is prevalent worldwide and has been a serious concern among customers and food manufactures. Species adulteration in food or feed products may also cause other serious problems for safety and health reasons such as species-associated pathogen contaminations and allergic reactions in sensitized individuals (Hsieh et al., 1999 (Reference 24)). There are also those who refrain from consuming these edible animal fats for health reasons because of their unhealthy fatty acid profile which have been implicated in such diseases as cancers, hypercholesterolemia, multiple sclerosis and coronary heart disease.

### Methods for Fat Species Identification and Fat Species Content Determination

The current global nature of the food trade with its intricate complexities has increased the potential for such fraudulent activities. The increased awareness among consumers regarding the ingredients used in the formulation of foods has made efforts by stakeholders (manufacturers, regulators, researchers and consumers) to authenticate the species origin of edible fats a priority.

There are also other reasons for which methods for fat speciation have been developed such as for authentication of fats used in feed formulation as a BSE control measure (Abbas et al. 2009 (Reference 1); Bellorini et al. 2005 (Reference 6)), for forensic purposes (Kagawa et al. 1996 (Reference 28); Moawad et al. 2009 (Reference 35)), and as an indirect approach for meat speciation (Chernukha 2011 (Reference 16)). However, determining the identity of edible animal fats in processed foods or composite blends is a difficult task as the adulterant has a composition similar to the original fat or oil. In the past years, many analytical methods have been reported in the literature for the identification of origin of the animal fat. They mainly include fat-based methods and DNA-based methods. Fat-based methods rely on subtle differences in the chemical (fatty acid composition and/or their positional distribution on the triacylglycerol (TAG) molecule) or physical (molecular structure and melting/crystallization temperatures) nature of different edible animal fats to identify their species origin while deoxyribonucleic acids (DNA)-based methods detects species-dependent differences at the gene level.

Fat-Based Methods for Species Identification

Using the fatty acid profile as a means for species identification of edible animal fat is a challenging task as the fatty acid composition is greatly influenced by the dietary fat intake. The situation is even compounded in recent times where the fatty acid composition of animal tissues can be modified; for example as in enrichment with omega-3 fatty acids (Wood et al. 2004 (Reference 54)). This notwithstanding, species-specific differences in the digestion process of dietary fats (Raclot T., Holm C. and Langin D. 2001. “Fatty acid specificity of hormone-sensitive lipase.” Implication in the selective hydrolysis of triacylglycerols. J. Lipid Res. 42(12):2049-2057 (2001) and Sato K., Suzuki K. and Akiba Y. “Species differences in substrate specificity of lipoprotein lipase purified from chickens and rats.” Comp. Biochem. Physiol. A. Mol. Integr. Physiol. 119(2):569-573 (1998)) and the different nutrient demands of divergent species (which is ultimately reflected in the composition of the deposited lipids) (Kagawa et al. 1996 (Reference 28); Schreiner et al. 2006 (Reference 47)), have been exploited for species identification of fat. Typically, the fat is removed by saponification, converted to methyl esters, and the fatty acid (FA) pattern is analyzed by various techniques such as gas chromatography (GC), high performance liquid chromatography (HPLC), Fourier transform infrared (FIT) spectroscopy and near infrared (NIR). These techniques are almost often combined with chemometric techniques as principal component analysis (PCA) or linear discriminant analysis (LDA) to allow for the recognition of patterns from the large data sets typically generated by the use of such instruments. These methods although useful are laborious and require long testing times, require an experienced analyst and involve the use of expensive instruments. In addition, most of these methods tend to be effective only when the target is present in copious amounts. Besides interpretation of data is not clear-cut as different researchers have used different interpretations of the results to mean the same thing. Thus, alternative methods that are fast and low in cost for species identification of animal fat are highly desirable.

### DNA-Based Methods for Species Identification

More recently, DNA molecules have become target molecules for species identification in foods because of their high stability and also their presence in most biological tissues. Specific amplification of a fragment of DNA by means of polymerase chain reaction (PCR) with subsequent fragment size verification upon gel electrophoresis is the simplest DNA-based strategy for species identification of animal tissues. More species-specific variations such as restriction fragment length polymorphism (RFLP) PCR (Aida et al. 2005, 2007, 2011 (Reference 3, Reference 4 and Reference 5), analysis of single strand conformation polymorphism (SSCP) PCR, sequencing of fragments, and simultaneous amplification of two or more fragments with different primer pairs (multiplex PCR) have been developed for species identification of edible animal fats. With these DNA-based methods, mitochondrial DNA is generally the target as it has several advantages over nuclear DNA (Rastogi et al. 2007 (Reference 41)). These methods could be equally applied for species identification of meat and fat or other tissues because DNA is an universal biomarker in all biological tissues. Although DNA-based methods are useful and have been considered as a convincing method for speciation, the success of these DNA techniques is dependent on the amount and quality of DNA extracted from the sample. Several food processes have a negative influence on the accessibility and extraction of appropriate DNA material for PCR and hence renders DNA-based methods ineffective in certain instances. DNA is degraded by high temperature food processes either directly (Bellorini et al. 2005 (Reference 6)) or indirectly through the action of radicals furnished by Maillard products that are generated during the thermal processing (Hiramoto et al. 1994 (Reference 23). DNA may also be degraded during such food processes as hydrolysis (both enzymatic and chemical) and mechanical treatment (shear forces) (Jacobsen and Greiner 2002 (Reference 27)). Typically, DNA is not detectable in highly heat-processed food products, hydrolyzed products, and highly purified products (e.g. refined oils) (Kuiper 1999 (Reference 30)). In addition, DNA-based methods also require the use of major instruments, are prone to contamination, require highly technical skills, and are not feasible for large sample screening or rapid field testing. Both fat-based and DNA-based techniques have been shown useful for species identification of animal fat. However, besides the shortcomings of these methods mentioned above, these methods have focused almost exclusively on speciation of raw fat. Thus, although the usefulness of these methods for identifying the species origin of raw fat samples can be vouched for, the same cannot be said in situations in which these animal fats are present in processed foods.

### Protein-Based Methods for Species Identification

Protein-based immunoassays are based on the specific binding reaction between an antigen and the antibody. Immunoassays do not require major investment in equipment, are easy to perform, need only small quantities of test sample and immunoreagents, are amenable to field testing and have the capacity for large-scale screening. Immunoassays are therefore widely accepted by regulatory bodies as a quick and sensitive method for screening and monitoring substances in food and agricultural products. In addition, immunoassays can be performed in a complicated sample mixture without laborious isolation or purification of the target analyte(s) from the sample. If sufficient amount of soluble proteins can be extracted from the adipose tissue, the development of more convenient and rapid methods based on immunochemical principles for animal fat detection/speciation would be advantageous and desirable.

### Requirements for Protein-Based Immunoassay Development

The performance of the immunoassay rests primarily on the nature, quality, and availability of the detecting antibodies to capture the target protein antigen (analyte) in a sample extract. In order to develop an immunoassay for species identification of adipose tissue, it is necessary that a suitable antigen (usually a protein) biomarker be selected for the purpose. Although proteins are generally more heat-labile than DNA and most current immunoassays target native proteins, some proteins are highly stable and can be used as the antigen for antibody development and antibody recognition. The conditions of an ideal species marker should be that the antigen marker is present in the tissue in significant amount and is uniformly distributed throughout the tissue so that the detection result can be sensitive and representative, and that the binding between the antibody and the antigen is stable after heat processing so that cooking would not affect the immunoreactivity for the detection.

### Identified Proteins in Adipose Tissue

Adipose tissue or fat tissue is a kind of loose connective tissue composed of mature adipocytes, fibroblasts, immune cells, adipose tissue matrix and blood vessels. Approximately 60 to 85% of the weight of adipose tissue is lipid with 90 to 99% of the lipid being triglyceride. The remaining weight of adipose tissue is composed of water (5 to 30%) and protein (2 to 3%) (Schaffler A., Scholmerich J. and Buehler C. “Mechanisms of Disease: adipocytokines and visceral adipose tissue-emerging role in intestinal and mesenteric diseases.” Nat. Clin. Pract. Gastroenterol. Hepatol. 2, 103-111 (2005)). Adipose tissue secretes different types of proteins that play important roles in homeostasis and metabolism through their autocrine, paracrine, and endocrine effects. The term adipokine has been suggested to describe all proteins secreted from any type of adipocyte (Trayhurn et al. 2011 (Reference 51)). Over the past century, proteins secreted from adipose tissue have been investigated. Physiologists have reported that a number of proteins, such as cytokines and cytokine-related proteins, chemokines, other immune-related proteins, proteins involved in the fibrinolytic system, complement and complement-related proteins for lipid metabolism or transport, and enzymes involved in steroid metabolism are secreted in adipose tissue (Kershaw and Flier 2004 (Reference 29); Rosenow et al. 2010 (Reference 44)). In addition, adipose tissue has also been shown to secrete contractile muscle proteins. For instance, muscle proteins including myosin, tropomyosin-2, tropomyosin α-3, and tropomyosin α-4 have been detected in human and porcine adipose tissues (Rosenow et al. 2010 (Reference 44); Ahmed et al. 2010 (Reference 2)).

### Troponin I as a Suitable Marker Protein for the Determination of Animal Species Origin of Adipose Tissue

It has been discovered that Troponin I (TnI) can be found in muscle-free adipose tissue in sufficient amount to allow TnI to be used a suitable species-marker protein for the species identification and species content determination of animal adipose tissue. TnI is a part of the muscle contractile protein, troponin which consists of three subunits, Troponin C (TnC), Troponin T (TnT) and Troponin I (TnI). TnI, the inhibitory subunit of the Troponin complex, consists of a family of three muscle-specific myofibrillar proteins involved in the calcium-sensitive regulation of contraction in both skeletal and cardiac muscle (Wilkinson and Grand 1978 (Reference 53)). TnI-skeletal-slow-twitch (TnI1), TnI-skeletal-fast-twitch (TnI2) and TnI-cardiac (Tn3) which are the individual members of this family, are encoded by separate genes in mammals and expressed differentially in various classes of muscle fibers (Yang et al. 2010 (Reference 55)). As TnI has been classified as muscle protein in the past years, the presence of TnI in adipose tissue has never been reported, however, the concept was indirectly supported by Yang et al. (2010) (Reference 56) who reported from their gene expression profiling studies that the TnI1 and TnI2 genes also to be expressed in many other tissues studied including porcine adipose tissue. Following are illustrations to demonstrate the presence of TnI in adipose tissues of pig, cattle and chicken.

In one embodiment of the present invention, the antibodies used to detect TnI may be present on a sensor (biosensor or immunosensor) and the signal can be detected electronically or in many other ways such as by an optical fiber, etc.

In one embodiment, the present invention may employ variations of immunoassay (enzyme immunoassay, fluorescent immunoassay, radioisotope immunoassay, chemiluminescent immunoassay, etc.) automation. The automation of immunoassays has been popular in laboratories for high throughput screening routing tests. An advantage of immunoassay automation is that every procedure of various immunoassays can be operated by the automated instrument, not manually once the sample extracts have been prepared. The lateral flow strip test (an immunochromatographic method) can be read visually or by using a digitized device, such as a handheld type for field use, to obtain semi-quantitative readings.

### EXAMPLES

### Example 1

This example demonstrates that substantial proteins can be extracted from adipose tissue and compared with proteins extracted from lean muscle tissue. Soluble proteins are extracted from trimmed muscle-free adipose tissues as well as from lean muscle tissues of pig, cattle and chicken, raw and cooked, by using a 1:1 mixture of petroleum ether and 0.5 M NaCl solution with (hot) or without (cold) a heat treatment (heating the homogenate at a boiling water bath for 15 min) The protein concentrations are compared in these extracts (Table 1). Although muscle contains much more (4.7-41.5 folds) soluble proteins than that in the adipose tissue, substantial amount of proteins can still be extracted from the fat tissue. These soluble proteins can be candidates serving as a fat tissue marker for antibody development. Table 1 shows total soluble proteins extracted from raw (r) and cooked (c) fat (F) and meat (M) samples of pig (P), cattle (B) and chicken (C) using two extraction conditions, cold (o) and hot (h). For example, “PFro” is an extract for a pig fat sample that is raw and cold.

TABLE 1

| | | | | Ration of |
|---|---|---|---|---|
| | Protein | Meat | Protein | protein |
| Fat Sample | concentration | Sample | concentration | concentrations |
| Extract | (mg/g) | Extract | (mg/g) | (fat:meat) |
| PFro | 7.61 ± 0.14 | PMro | 47.34 ± 1.46 | 1:6.2 |
| PFco | 0.13 ± 0.01 | PMco | 3.71 | 1:28.5 |
| PFrh | 0.45 ± 0.02 | PMrb | 3.1 ± 0.13 | 1:6.9 |
| PFch | 0.32 ± 0.01 | PMch | 1.7 ± 0.05 | 1:5.3 |
| BFro | 5.66 ± 0.42 | BMro | 49.43 ± 1.29 | 1:8.7 |
| BFco | 0.23 ± 0.02 | BMco | 3.3 ± 1.1 | 1:14.4 |
| BFrh | 0.58 ± 0.03 | BMrh | 3.96 ± 0.16 | 1:6.8 |
| BFch | 0.27 ± 0.01 | BMch | 2.75 ± 0.18 | 1:10.2 |
| CFro | 14.43 ± 2.97 | CMro | 67.87 ± 4.71 | 1:4.7 |
| CFco | 0.26 ± 0.04 | CMco | 10.8 ± 0.02 | 1:41.5 |
| CFrh | 0.85 ± 0.05 | CMrh | 6.2 ± 0.13 | 1:7.3 |
| CFch | 0.53 ± 0.09 | CMch | 5.44 ± 0.10 | 1:10.3 |

### Example 2

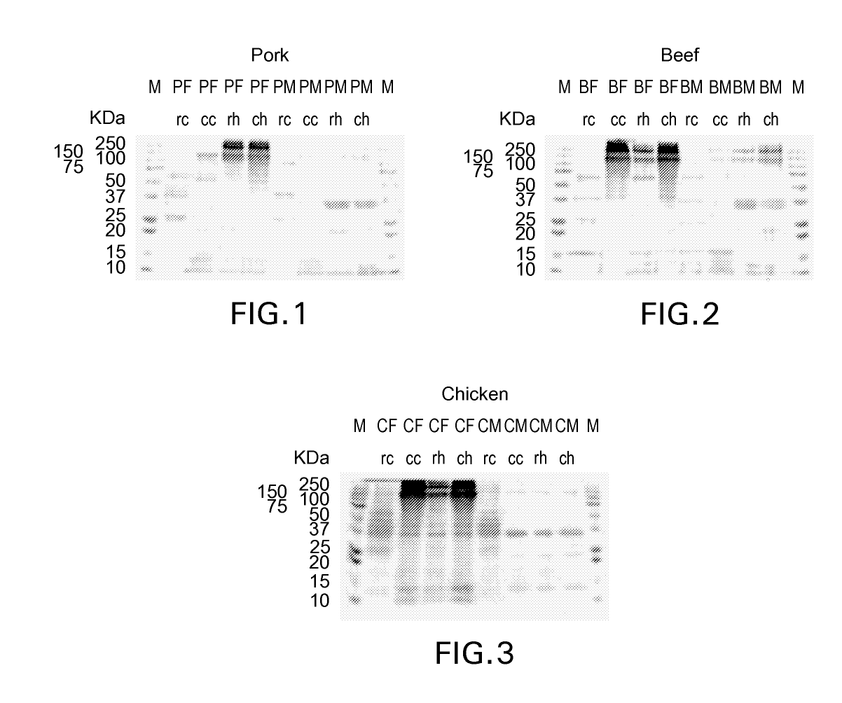

Comparing the soluble protein profile in the extracts of raw and cooked fat and meat samples by SDS-PAGE The results showed that adipose tissue contains more high molecular weight proteins than muscle tissue. Cooking or heating eliminates most of the heat-labile proteins resulting in less protein bands on the gel (FIGS. 1, 2 and 3). FIGS. 1, 2 and 3 show an SDS-PAGE protein profile of cooked and raw pork (FIG. 1), beef (FIG. 2), and chicken (FIG. 3) meat and fat extracts with and without heating treatment. Lane M in FIGS. 1, 2 and 3 is the protein molecular weight marker.

### Example 3

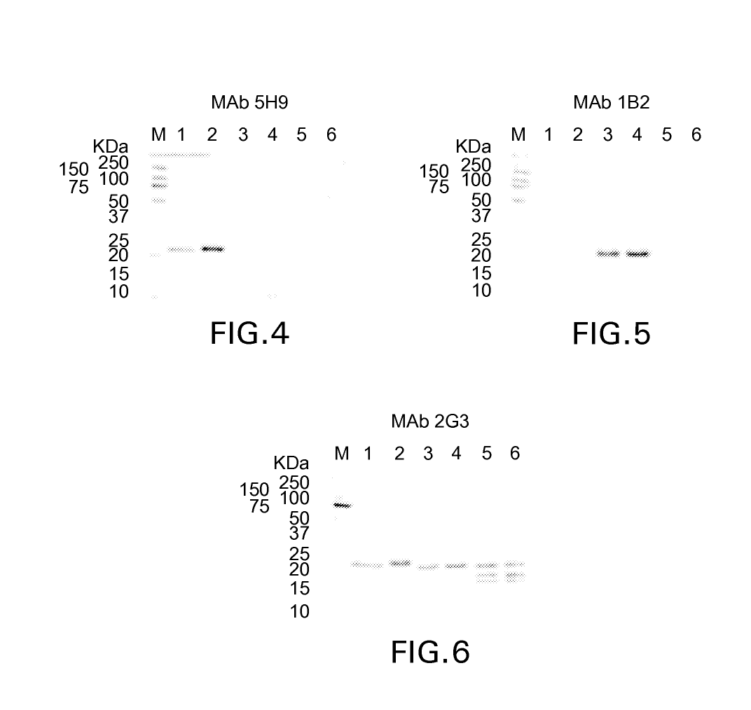

In this example, species-specific TnI present in adipose tissue is probed by species-specific anti-TnI monoclonal antibodies using western blot analysis. In previous studies, a panel of species-specific anti-TnI monoclonal antibodies (MAbs) have been produced. Three MAbs are selected in this experiment to probe the presence of TnI in the protein extracts of raw and cooked adipose tissue and lean muscle tissue from pig, cattle and poultry using western blot. MAb 5H9 is specific to pork skeletal muscle TnI (Chen et al. 1998 (Reference 13; Chen and Hsieh 2002 (Reference 14)). MAb IB2 is specific to bovine and ovine TnI (Chen et al. 2004 (Reference 15)), MAb 2G3 can recognize TnI in all animal species tested including porcine, bovine, sheep, horse, deer, chicken, turkey, duck, goose, ostrich and catfish. (Chen et al, 2002 (Reference 14)). Western blot results (FIGS. 4, 5 and 6) reveal that MAb 5H9 (pork TnI specific) only recognizes pork fat (Lane 1) and pork muscle (Lane 2) but not beef or chicken tissues; MAb 1B2 recognizes beef fat (Lane 3) and beef meat (Lane 4); while MAb 2G3 recognizes TnI in all animal species. Results indicate that each antibody not only reacts with the same antigen (TnI) in both adipose tissue and in lean muscle tissue but also binds to the respective species-specific region on the TnI peptide. In addition TnI is clearly present in adipose tissue in significant amount, although less than the amount in muscle tissue.

FIGS. 4, 5 and 6 are western blots showing the species-specificity of MAbs 5H9 (FIG. 4), 1B2 (FIG. 5), 2G3 (FIG. 6) for meat and fat extracts from different species. In FIGS. 4, 5 and 6, lane M is for a protein molecular weight marker, lane 1 is for PFrh, lane 2 is for PMco, lane 3 is for BFrh, lane 4 is for BMco, lane 5 is for CFrh and lane 6 is for CMco.

### Example 4

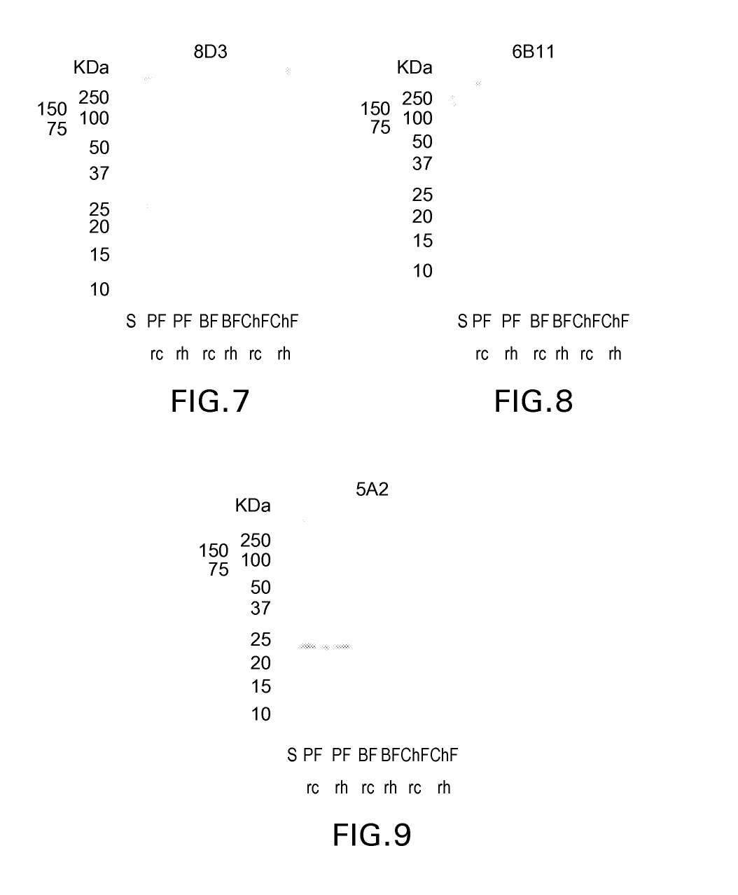

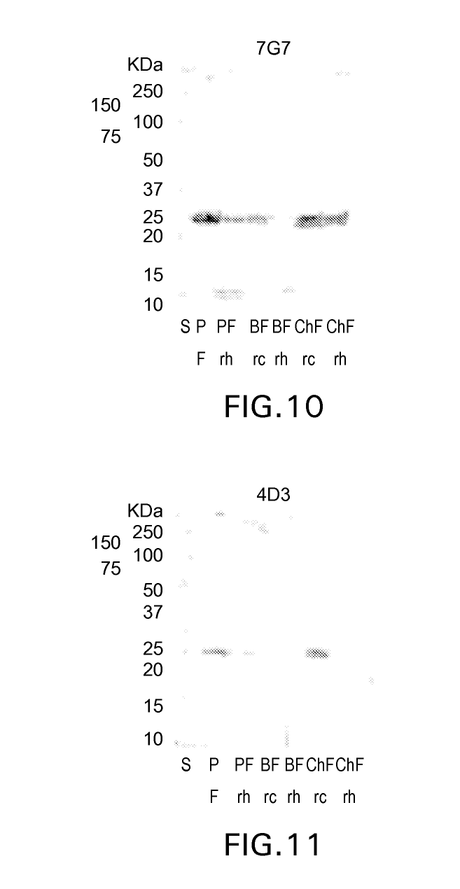

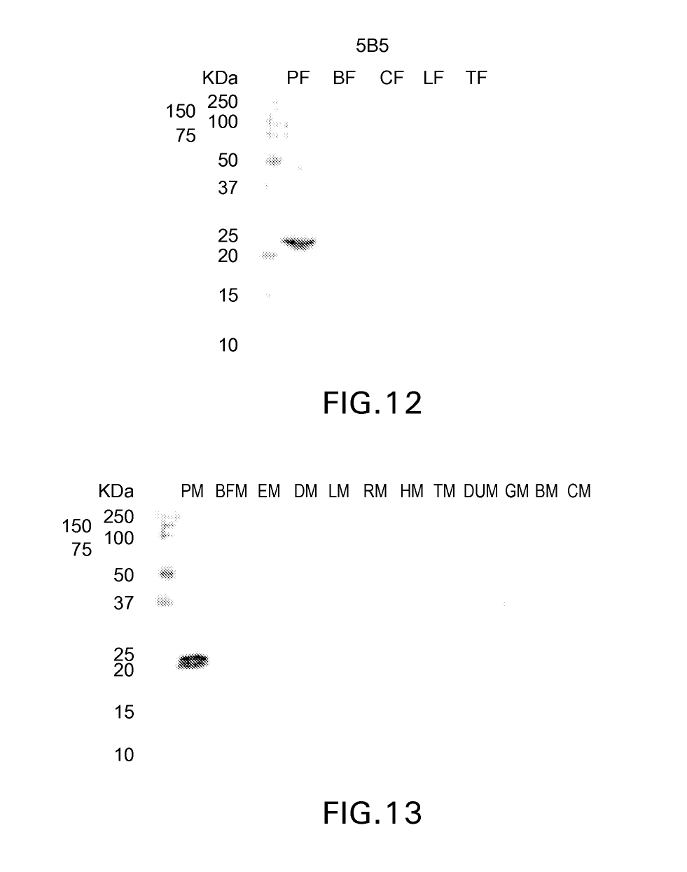

This example shows that TnI is the most antigenic protein in the extract of adipose tissue. This experiment addresses the following two major points: (1) anti-porcine fat antibodies can be developed by raising against the crude thermal-stable proteins from the extracts of cooked porcine adipose tissue, and (2) the antigenic protein of these newly developed MAbs is proven to be TnI. Subsequently, a panel of MAbs are developed using partially purified crude protein from cooked porcine adipose tissue as the immunogen in order to reveal all possible antigenic proteins which illicited the MAbs production. A total of 6 MAbs were cloned and used for this experiment. They are 4D3 (IgGI), 5A2 (IgG3), 5B5 (IgG2a), 6BII (IgG1), 707 (IgG1) and 8D3 (IgG3). All of these MAbs react to a 23 KDa antigenic protein (FIGS. 7, 8, 9, 10, 11, 12 and 13) indicating this 23 Kda protein is heat-stable and most antigenic in the adipose tissue. FIGS. 12 and 13 demonstrate the strong porcine-specificity of MAb 5B5 when it was screened against the protein extracts of fat and lean meat from other animal species using western blot. MAb 5B5 can react with the same 23 KDa antigen in both adipose tissue and lean meat tissue. Furthermore, to verify the 23 KDa antigenic protein to be TnI, an inhibition test is performed using the previously developed porcine TnI specific MAb 5H9 against all above 6 new MAbs. All of these new MAbs showed inhibitive binding with MAb5H9 and they also inhibited each other with the “Additive Index” below 50% indicating all of these MAbs bind to the same antigen which is the same antigen (TnI) of MAb5H9. It is considered that the antibodies share the same binding side or their binding sides overlap to some degree if AI is below 50%. (Friguet, et al. 1983 (Reference 21)). These porcine-specific MAbs can be used individually or in combination for future development of suitable immunoassays or pork fat detection. Likewise, other species-specific anti-TnI antibodies can also be developed and/or used for species identification of adipose tissue of other species such as bovine and poultry, as demonstrated in FIGS. 4, 5 and 6.

FIGS. 7, 8, 9, 10 and 11 are western blot results that that the antigenic proteins of all 5 MAbs which was raised against crude heated protein extracted from porcine adipose tissue are of 23 kDa and later it is identified as troponin I indicating troponin I is the most antigenic protein in the crude fat tissue protein extract. Results also indicate that among the five MAbs, MAbs 8D3 and 5A2 are porcine specific. Others also cross-reacted with protein extracts from bovine and chicken fat tissue. S=molecular weight standards; PF=porcine fat; BF=bovine fat; and ChF=chicken fat.

In FIGS. 12 and 13 Antigenic protein in fat extracts (FIG. 12) and in meat extracts (FIG. 13) is revealed by western blot analysis based on MAb 5B5. This MAb recognizes 23 kDa protein only in the extract from pork fat or meat but not in other animal species. In FIGS. 12 and 13: PF=pork fat; BF=beef fat; CF=chicken fat; LF=lamb fat; TF=turkey fat; PM=pork meat; BFM=buffalo meat, EM=elk meat; DM=deer meat; LM=lamb meat; RM=rabbit meat; HM=horse meat; TM=turkey meat; DUM=duck meat; OM=goose meat; BM=beef meat; CM=chicken meat.

### CONCLUSION

The presence of TnI has been identified in adipose tissue. The extraction of this protein from fat tissue was enhanced by a heat treatment. The existence of TnI in adipose tissue was confirmed by western blot based on three MAbs that are specific to pork, beef, and all animals TnI. The results obtained provided solid evidence of the existence of TnI in adipose tissue. In addition, TnI potentially serves as an efficient species marker for the detection of fat species in meat and food products via immunoassay techniques. The identified antigenic proteins of 6 MAbs produced by immunizing animal with crude heat-stable proteins extracted from adipose tissue are all proved to be TnI indicating its strong antigenicity. Based on these results, a rapid and reliable tool based on the immunochemical detection of TnI may be developed for the speciation and detection of target species of fat content in processed fat or meat products.

### REFERENCES

The following references are referred to above and/or describe technology that may be used with the present invention and are incorporated herein by reference:
1. O. Abbas, J. A. F. Pierna, R. Codony, C. von Holst, and V. Baeten, “Assessment of the discrimination of animal fat by FT-Raman spectroscopy,” J. Mol. Struct. 924-26:294-300 (2009).
2. M. Ahmed, M. J. Neville, M. J. Edelmann, B. M. Kessler, and F. Karpe, “Proteomic analysis of human adipose tissue after rosiglitazone treatment shows coordinated changes to promote glucose uptake,” Obesity (Silver Spring) 18(1):27-34 (2010).
3. A. A. Aida, Y. B. C. Man, C. M. Wong, A. R. Raha, and R. Son, “Analysis of raw meats and fats of pigs using polymerase chain reaction for Halal authentication,” Meat Sci. 69(1):47-52 (2005).
4. A. A. Aida, Y. B. C. Man, A. A. Hassan, A. R. Raha, and R. Son, “Specific polymerase chain reaction (PCR) analysis of raw meats and fats of pigs for halal authentication,” Middle East Journal of Scientific Research 7(6): 1008-13 (2011).
5. A. A. Aida, Y. B. C. Man, A. R. Raha, and R. Son, “Detection of pig derivatives in food products for halal authentication by polymerase chain reaction-restriction fragment length polymorphism,” J. Sci. Food Agr. 87(4):569-72 (2007).
6. S. Bellorini, S. Strathmann, V. Baeten, O. Fumiere, G. Berben, S. Tirendi, and C. von Holst, “Discriminating animal fats and their origins: assessing the potentials of Fourier transform infrared spectroscopy, gas chromatography, immunoassay and polymerase chain reaction techniques,” Anal. Bioanal. Chem. 382(4):1073-83 (2005).
7. E. G. Bligh and W. J. Dyer, “A rapid method of total lipid extraction and purification,” Can. J. Biochem. Physiol. 37(8):911-17 (1959).
8. S. Boulant, R. Montserret, R. G. Hope, M. Ratinier, P. Targett-Adams, J. P. Lavergne, F. Penin, and J. McLauchlan, “Structural determinants that target the hepatitis C virus core protein to lipid droplets,” J. Biol. Chem. 281(31):22236-47 (2006).
9. C. L. Brennan, M. Hoenig, and D. C. Ferguson, “GLUT4 but not GLUT1 expression decreases early in the development of feline obesity,” Domest. Anim. Endocrin. 26(4):291-301 (2004).
10. Y. B. C. Man, H. L. Gan, I. NorAini, S. A. H. Nazimah, and C. P. Tan, “Detection of lard adulteration in RBD palm olein using an electronic nose,” Food Chem. 90(4):829-35 (2005).
11. Y. B. C. Man, and M. E. S. Mirghani, “Detection of lard mixed with body fats of chicken, lamb, and cow by Fourier transform infrared spectroscopy,” Journal of the American Oil Chemists Society 78(7):753-761 (2001).
12. Y. B. C. Man, Z. A. Syahariza, and A. Rohman, “Discriminant analysis of selected edible fats and oils and those in biscuit formulation using FTIR spectroscopy,” Food Anal. Methods 4(3):404-09 (2011).
13. F-C. Chen, Y-H. P. Hsieh, and R. C. Bridgman, “Monoclonal antibodies to porcine thermal-stable muscle protein for detection of pork in raw and cooked meats,” J. Food Sci. 63: 201-05 (1998).
14. F-C. Chen, and Y-H. P. Hsieh, “Porcine troponin I: a thermostable species marker protein,” Meat Sci. 61(1):55-60 (2002).
15. F-C. Chen Y-H. P. Hsieh, R. C. Bridgman, “Monoclonal antibody-based sandwich enzyme-linked immunosorbent assay for sensitive detection of prohibited ruminant proteins in feedstuffs,” J. Food Prot. 67:544-49 (2004).
16. I. Chernukha, “Comparative study of meat composition from various animal species,” International 56th Meat Industry Conference. Tara, Serbia: technologija mesa, 167-71 (2011).
17. S. T. Chin, Y. B. C. Man, C. P. Tan, and D. M. Hashim, “Rapid Profiling of Animal-Derived Fatty Acids Using Fast GC x GC Coupled to Time-of-Flight Mass Spectrometry,” Journal of the American Oil Chemists Society 86(10):949-58 (2009).
18. B. B. De Taeye, C. Christophe Morisseau, J. Coyle, J. W. Covington, A. Luria, J. Yang, S. B. Murphy, D. B. Friedman, B. B. Hammock, and D. E. Vaughan, “Expression and regulation of soluble epoxide hydrolase in adipose tissue, Obesity 18:489-498 (2010).
19. P. Dugo, T. Kumm, A. Fazio, G. Dugo, and L. Mondello, “Determination of beef tallow in lard through a multidimensional off-line non-aqueous reversed phase-argentation LC method coupled to mass spectrometry,” J. Sep. Sci. 29(4):567-75 (2006).
20. ECSSC, Opinion on the safety of tallow derivatives from cattle tallow (1999).
21. B. Friguet, L. Djavadi-Ohaniance, J. Pages, A. Bussard, and M. Goldberg, “A convenient enzyme-linked immunosorbent assay for testing whether monoclonal antibodies recognize the same antigenic site. Application to hybridomas specific for the beta 2-subunit of Escherichia coli tryptophan synthase,” J. lmmunol. Methods 60:351-58 (1983).
22. G. Gondret, B. Guevel, E. Com, A. Vincent, and B. Lebret, “A comparison of subcutaneous adipose tissue proteomes in juvenile piglets with a contrasted adiposity underscored similarities with human obesity,” J. Proteomics 75(3):949-61 (2012).
23. K. Hiramoto, K. Kido, and K. Kikugawa, “DNA Breaking by Maillard Products of Glucose Amino-Acid Mixtures Formed in an Aqueous System,” J. Agr. Food Chem. 42(3):689-94 (1994).
24. Y-H. P. Hsieh, F-C. Chen, and N. Djurdjevic, “Monoclonal antibodies against heat-treated muscle proteins for species identification and end-point cooking temperature determination of cooked meats,” Quality Attributes of Muscle Foods. Xiong, Ho and Shahidi (eds.). Kluwer Academic/Plenum Publishers, N.Y. 287-306 (1999).
25. G. Iacobellis, D. Corradi, and A. M. Sharma, “Epicardial adipose tissue: anatomic, biomolecular and clinical relationships with the heart,” Nat. Clin. Pract. Cardiovasc. Med. 2(10):536-43(2005).
26. D. Indrasti, Y. B. C. Man, S. Mustafa, and D. M. Hashim, “Lard detection based on fatty acids profile using comprehensive gas chromatography hyphenated with time-of-flight mass spectrometry,” Food Chem. 122(4):1273-77 (2010).
27. H-J. Jacobsen and R. Greiner, “Methods for detecting genetic manipulation in grain legumes,” Jackson, J. F. & Linskens, H. F., editors. Molecular methods of plant analysis: Testing for genetic manipulation in plants New York: Springer, 64 (2002).
28. M. Kagawa, K. Matsubara, K. Kimura, H. Shiono, and Y. Fukui, “Species identification by the positional analysis of fatty acid composition in triacylglyceride of adipose and bone tissues,” Forensic. Sci. Int. 79(3):215-26 (1996).
29. E. E. Kershaw, and J. S. Flier, “Adipose tissue as an endocrine organ,” J. Clin. Endocrinol. Metab. 89,2548-56 (2004).
30. H. A. Kuiper, “Summary report of the ILSI Europe workshop on detection methods for novel foods derived from genetically modified organisms,” Food Control 10(6):339-49 (1999).
31. A. Lazarev, G. Smejkal, I. Romanovsky, A. Kwan, H. Cao, G. S. Hotamisligil, and A. R. Ivanov, “Proteomic analysis of murine adipose tissue using pressure cycling technology and high resolution tandem mass spectrometry,” US HUPO 3rd Annual Conference, Seattle, Wash. (2007).
32. D. Lichtenberg, E. Opatowski and M. M. Kozlov, “Phase boundaries in mixtures of membrane-forming amphiphiles and micelle-forming amphiphiles,” Biochim. Biophys. Acta 1508(12):1-19 (2000).
33. J. M. N. Marikkar, H. M. Ghazali, Y. B. C. Man and O. M. Lai, “The use of cooling and heating thermograms for monitoring of tallow, lard and chicken fat adulterations in canola oil,” Food Research International 35 (10):1007-14 (2002).
34. J. M. N. Marikkar, H. M. Ghazali, Y. B. C. Man, T. S. G. Peiris and O. M. Lai, “Distinguishing lard from other animal fats in admixtures of some vegetable oils using liquid chromatographic data coupled with multivariate data analysis,” Food Chem. 91(1):5-14 (2005).
35. M. S. Moawad, M. A. Tony and H. A. Aref, “Forensic identification of subcutaneous and perirenal adipose tissue samples in some farm animals using gas liquid chromatography,” Mansoura. Vet. Med. J. XI(1): 13-20(2009).
36. N. H. Mohan, B. C. Sarmah, M. K. Tamuli, A. Das, and K. M. Bujarbaruah, “Electrophoretic profile of porcine adipose tissue and a method for extraction of soluble proteins from fat tissue,” Indian J. Anim. Sci. 77(12):1248-50 (2007).
37. J. F. Montiel-Sosa, E. Ruiz-Pesini, J. Montoya, P. Roncales, M. J. Lopez-Perez, and A. Perez-Martos, “Direct and highly species-specific detection of pork meat and fat in meat products by PCR amplification of mitochondrial DNA,” J. Agric Food Chem. 48(7):2829-32 (2000).
38. M. Motoyama, M. Ando M. K. Sasaki and H. O. Hamaguchi, “Differentiation of Animal Fats from Different Origins: Use of Polymorphic Features Detected by Raman Spectroscopy,” Appl. Spectrosc. 64(11):1244-50 (2010).
39. H.R. Mottram, Z. M. Crossman, and R. P. Evershed, “Regiospecific characterisation of the triacylglycerols in animal fats using high performance liquid chromatography atmospheric pressure chemical ionisation mass spectrometry,” Analyst 126(7):1018-1024 (2001).
40. D. J. Murphy, “The biogenesis and functions of lipid bodies in animals, plants and microorganisms,” Prog. Lipid Res. 40(5):325-438 (2001).
41. G. Rastogi, M. S. Dharne, S. Walujkar, A. Kwnar, M. S. Patole, and Y. S. Shouche, “Species identification and authentication of tissues of animal origin using mitochondrial and nuclear markers,” Meat Sci. 76(4):666-74 (2007).
42. A. Rohman and Y. B. C. Man, “FTIR spectroscopy combined with chemometrics for analysis of lard in the mixtures with body fats of lamb, cow, and chicken,” International Food Research Journal 17(3):519-26 (2010).
43. A. Rohman, Y. B. C. Man, P. Hashim and A. Ismail, “FTIR spectroscopy combined with chemometrics for analysis of lard adulteration in some vegetable oils,” Journal of Food 9(2):96-101 (2011).
44. A. Rosenow, T. N. Arrey, F. G. Bouwman, J. P. Noben, M. Wabitsch, E. C. M. Mariman, M. Karas, and J. Renes, “Identification of Novel Human Adipocyte Secreted Proteins by Using SGBS Cells,” J. Proteome Res. 9(10):5389-5401 (2010).
45. T. Sajic, G. Hopfgartner, I. Szanto, and E. Varesio, “ Comparison of three detergent-free protein extraction protocols for white adipose tissue,” Anal. Biochem. 415(2):215-217.
46. A. Salgado-Somoza, E. Teijeira-Fernandez, A. L. Fernandez, J. R. Gonzalez-Juanatey, and S. Eiras, “Proteomic analysis of epicardial and subcutaneous adipose tissue reveals differences in proteins involved in oxidative stress,” Am. J. Physiol. Heart Circ. Physiol. 299(1):H202-209 (2010).
47. M. Schreiner, R. G. Moreira and H. W. Hulan, “Positional distribution of fatty acids in egg yolk lipids,” J. Food Lipids 13(1):36-56 (2006).
48. A. M. Seddon, P. Curnow and P. J. Booth, “Membrane proteins, lipids and detergents: not just a soap opera,” Biochim. Biophys. Acta 1666(1-2): 105-117 (2004).
49. Sucipto, Irzaman, T. T. Irawadi, and A. M. Fauzi, “Potential of conductance measurement for lard detection,” International Journal of Basic and Applied Sciences 11(5):26-30 (2011).
50. A. Szab\acute{0}, H. Febel, L. Sugar, and R. Romvari, “Fatty acid regiodistribution analysis of divergent animal triacylglycerol samples—a possible approach for species differentiation,” Journal of Food Lipids 14(1):62-77 (2007).
51. P. Trayhurn, C. A. Drevon and J. Eckel, “Secreted proteins from adipose tissue and skeletal muscle—adipokines, myokines and adipose/muscle cross-talk,” Arch. Physiol. Biochem. 117(2):47-56 (2011).
52. L. Vaclavik, V. Hrbek, T. Cajka, B.A. Rohlik, P. Pipek, and J. Hajslova, “Authentication of animal fats using direct analysis in real time (DART) ionization-mass spectrometry and chemometric tools,” J. Agric. Food Chem. 59(11):5919-26 (2011).
53. J. M. Wilkinson and R. J. Grand, “Comparison of amino acid sequence of troponin I from different striated muscles,” Nature 271(5640):31-35 (1978).
54. J. D. Wood, R. I. Richardson, G. R. Nute, A. V. Fisher, M. M. Campo, E. Kasapidou, P. R. Sheard, and M. Enser, “Effects of fatty acids on meat quality: a review,” Meat Sci. 66(1):21-32 (2004).
55. H. Yang, Z. Y. Xu, M. G. Lei, F. E. Li, C. Y. Deng, Y. Z. Xiong, and B. Zuo, “Association of 3 polymorphisms in porcine troponin I genes (TNNI1 and TNNI2) with meat quality traits,” J. Appl. Genet. 51(1):51-57 (2010).
56. H. Yang, Z. Xu, Z. Ma, Y. Xiong, C. Deng, and B. Zuo, “Molecular cloning and comparative characterization of the porcine troponin I family,” Anim. Biotechnol. 21(1):64-76 (2010).
57. R. Zasadny and K. Kwiatek, “Validation study of a new procedure for measuring insoluble impurities in animal fat,” J. Anim. Feed Sci. 15(2):337-44 (2006).
58. Y. T. Zhou, Z. W. Wang, M. Higa, C. B. Newgard, and R. H. Unger, “Reversing adipocyte differentiation: implications for treatment of obesity,” Proc. Mid Acad. Sci. USA 96(5):2391-95 (1999).

Having described the many embodiments of the present invention in detail, it will be apparent that modifications and variations are possible without departing from the scope of the invention defined in the appended claims. Furthermore, it should be appreciated that all examples in the present disclosure, while illustrating many embodiments of the invention, are provided as non-limiting examples and are, therefore, not to be taken as limiting the various aspects so illustrated.

While the present invention has been disclosed with references to certain embodiments, numerous modification, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method comprising the following steps:
(a) determining a species of a sample of adipose tissue based on a species of troponin I (TnI) detected in the sample of adipose tissue, and
(b) displaying the species of the sample of adipose tissue determined in step (a) to a user on a visual display device and/or saving the species of the sample of adipose tissue determined in step (a) to a storage medium.

2. The method of claim 1, wherein the method comprises the following step:
(c) detecting the species of TnI for the sample of adipose tissue.

3. The method of claim 1, wherein step (a) is conducted by a computer.

4. The method of claim 3, wherein the computer is an immunoassay system.

5. The method of claim 1, wherein step (b) is conducted by a computer.

6. The method of claim 5, wherein the computer is an immunoassay system.

7. The method of claim 1, wherein the species of the sample of adipose tissue determined in step (a) is displayed to the user on a visual display device.

8. The method of claim 1, wherein the species of the sample of adipose tissue determined in step (a) is saved to a storage medium.

9. The method of claim 1, wherein the species of the sample of adipose tissue is determined to be a porcine species in step (a).

10. The method of claim 1, wherein the species of the sample of adipose tissue is determined to be a poultry species in step (a).

11. The method of claim 1, wherein the species of the sample of adipose tissue is determined to be a ruminant species in step (a).

12. The method of claim 11, wherein the species of the sample of adipose tissue is determined to be a bovine species in step (a).

13. The method of claim 11, wherein the species of the sample of adipose tissue is determined to be a sheep species in step (a).
